(19) United States
(12) Patent Application Publication
(10) Pub. No.: US 2003/0096791 A1
(43) Pub. Date: May 22, 2003

(54) Taste masking of highly water-soluble drugs

(75) Inventors: Vijay V. Gupte, Vernon Hills, IL (US); Walid Habib, Crystal, MN (US); Kris E. Holt, Maple Grove, MN (US); Rajendra K. Khankari, Maple Grove, MN (US); John Hontz, Plymouth, MN (US)

Correspondence Address: Lerner, David, Littenberg,, Krumholz & Mentlik, LLP, 600 South Avenue West, Westfield, NJ, 07090, US

(73) Assignee: Cima Labs Inc., Eden Prairie, MN

(21) Appl. No.: 10/158,715

(22) Filed: May 30, 2002

Related U.S. Application Data
(60) Provisional application No. 60/294,938, filed on May 31, 2001.
(60) Provisional application No. 60/295,002, filed on Jun. 1, 2001.

(51) Int. Cl. A61K 31/716; A61K 47/00; A61K 9/14

(52) U.S. Cl. 514/57; 424/486; 424/488; 514/772.3; 514/781

(57) ABSTRACT

A taste-masked formulation which provides for effective taste-masking of an active ingredient such as cetirizine hydrochloride, while simultaneously providing a desired dissolution profile.

The present application claims the benefit of U.S. Provisional Application No. 60/294,938, filed on May 31, 2001 and U.S. Provisional No. 60/295,002, filed on Jun. 1, 2001, the contents of both are hereby incorporated by reference.

### BACKGROUND OF THE INVENTION

The active ingredients formulated into drug products often have an unpleasant taste. As the solubility of the active ingredient in saliva increases, so too does its offensive taste.

There are a variety of methods and formulas generally utilized in taste-masking. These include the use of flavors, sweeteners, effervescent systems and various coating strategies. However, for certain drugs, and in particular, antihistamines, such as cetirizine hydrochloride, traditional taste-masking methods have often proven ineffective.

Other prior taste-masking strategies, while effective in certain instances, can adversely affect dissolution times in the patient's mouth and/or stomach. Such strategies therefore may not suit the demands of rapidly dissolvable in-mouth oral dosage forms and other dosage forms such as those which are intended to promptly release an active ingredient after ingestion.

Modified celluloses such as hydroxypropylmethyl cellulose (“HPMC”), ethylcelluloses and mixtures of celluloses have been used to produce enteric coatings as well as coatings which can provide a controlled release of an active ingredient. Some of these coatings have also been used in taste-masking.

However, even the use of celluloses, as a single coating layer, was ineffective in taste-masking certain orally disintegrating tablets containing particularly offensive tasting water-soluble active ingredients such as, cetirizine hydrochloride. Therefore, there still remains a need for coatings which provides both effectively taste-masking and good release profile for the active ingredients, particularly, for use with highly water-soluble drugs such as cetirizine hydrochloride.

### SUMMARY OF THE INVENTION

One aspect of the present invention is directed to a taste-masked formulation which will reduce or eliminate the release of active ingredient in the mouth and yet will rapidly release the active ingredient in acidic conditions, such as those found in the stomach. These formulations include a taste-masked particles which themselves include (a) a predetermined amount of a particulate active ingredient; (b) at least one coating layer coating the particulate active ingredient.

These taste-masked, coated active ingredient particles might be used alone or blended with other active ingredients and/or pharmaceutically acceptable excipients to produce a taste-masked formulation. Other aspects of the present invention include methods of preparing taste-masked formulations, methods of using these formulations as well as the taste-masked formulations themselves.

The taste-masked formulations of the present invention preferably have slow release rates in conditions normally found in the mouth, such as less than 20% of the active ingredient in the formulation released in basic conditions within three minutes, and fast release rates under conditions normally found in the stomach, such as at least 65% of the active ingredient released in acidic pH within thirty minutes.

### DETAILED DESCRIPTION OF THE INVENTION

The present invention provides for a taste-masked formulation including taste-masked particles. The taste-masked particles include (1) at least one particulate active ingredient and (2) at least a taste-masking layer surrounding the at least one active ingredient. The taste-masking layer includes predetermined amount of a water-soluble polymer and a water-insoluble polymer, wherein the taste-masked formulation releases less than about 20% of the at least one active ingredient in an aqueous solution at a neutral to basic pH in about 3 minutes and releases at least about 65% of the at least one active ingredient at an acidic pH in about 30 minutes. If more than one layer is used to coat the active ingredient, then, preferably at least the outer layer will have such properties. Alternatively, the layers used, when taken in combination, will have such properties. In such instances, each of the taste-making layers will have the properties of delayed release of active ingredient in the mouth and rapid release in the stomach, or under such conditions as found therein, and each will contain a mixture of water-soluble and water-insoluble materials, although the specific materials used, and/or their relative proportions, may vary from layer to layer. Other layers which are not intended for taste-masking, such as a spacer layer or a layer which prevents reaction between the taste-masking coating and the active ingredient, can also be added. So long as at least are taste masking layer as defined herein is present. Preferably, the taste-masked formulations release less than about 16%, more preferably less than about 10%, of the at least one active ingredient in an aqueous solution at a basic pH in about 3 minutes and releases at least about 75%, more preferably at least about 85%, of the at least one active ingredient at an acidic pH in about 30 minutes.

In one preferred embodiment of the invention, the active ingredient is in the form of particles. As used herein, “active ingredient particles,” “particulate active ingredients” and the like include, but are not limited to particles, powders, adsorbents, granules, beads, or spheres of the active ingredient alone, in the presence of, in combination with, absorbed in or coated onto a solid support which is preferably in the form of particles, powders, adsorbents, granules, beads, or spheres. Spheres are preferred and particularly useful spheres include sugar spheres, microcrystalline cellulose spheres. Preferably, the average diameter of the solid support is, if any is used, from about 5 micrometers to about 280 micrometers, and more preferably from about 100 micrometers to about 200 micrometers. Generally, sufficient solid support particles are provided so as to allow the entire amount of the active ingredient to be layered, absorbed, adsorbed or applied thereto. Preferably, the weight ratio of active ingredient and solid support is from about 2:1 to about 1:20, more preferably from about 1.5:1 to about 1:10 and even more preferably from 1.2:1 to about 1:5.

In a further preferred embodiment of the present invention, a binder can be included to facilitate the coating of the active ingredient to the solid support. Preferably, the binder is selected from the group consisting of: hydroxypropyl methylcellulose, HPMC, polyvinyl pyrrolidone, starch, gum arabic, carboxymethylcellulose, polyvinylpyrrolidone, hydroxypropylcellulose, crystalline cellulose, sucrose, D-mannitol, dextrin, hydroxypropyl cellulose, hydroxypropylmethyl cellulose, polyvinylpyrrolidone, povidone, acacia, tragacanth, gelatin, cellulose materials, such as methyl cellulose and sodium carboxy methyl cellulose, alginic acids and salts thereof, magnesium aluminum silicate, polyethylene glycol, guar gum, polysaccharide acids, bentonites, sugars, and invert sugars, and the like. Generally, the binder is provided in an amount sufficient to facilitate adhesion of the entire quantity of active ingredient to the solid support. It can constitute up to about 5% by weight of the coated active ingredient particles, preferably up to about 2% and more preferably up to about 1%.

In a preferred embodiment, the taste-masking layer(s) has/have from about 2% to about 20% of a water-soluble polymer and from about 80% to about 98% of a water-insoluble polymer based on the total weight of the taste-masking layer, preferably from about 5% to about 18% of a water-soluble polymer and about 82% to about 95% of a water-insoluble polymer. More preferably, a taste-masking layer includes from about 8% to about 15% of a water-soluble polymer and from about 85% to about 92% of a water-insoluble polymer. Preferably, the water-soluble polymers contemplated for use in the present application include, but are not limited to, hydroxypropyl methyl cellulose, polyvinyl pyrrolidone, polyvinyl alcohol, hydroxypropyl cellulose, hydroxyethyl cellulose, methyl cellulose, carboxymethyl cellulose, and polyethylene glycols. Most preferably, the water-soluble polymer selected is HPMC commercially known as “Opadry®.” The water-insoluble polymers contemplated for use in the present application include, but are not limited to ethyl cellulose, hydroxypropyl methyl cellulose phthalate, polyvinyl acetate phthalate, hydroxypropyl methyl cellulose succinate, and shellac. Most preferably, the water-insoluble polymer selected is ethylcellulose commercially known as “Surelease®.” The taste-masking layer(s) is/are coated onto the active ingredient particles to produce “the coated active ingredient particles.”

The present invention is not limited to any thickness or composition of the coating layer(s). Any thickness and any combination of water-soluble polymers or water-insoluble polymers can be used as long as the coating layer meets the performance criteria, such as the desired taste-masking and the desired dissolution profile. Typically, the thickness of the coating layer can be defined in terms of the weight gain, e.g., a coating layer thickness of 30% refers to a weight gain of 30% by coating the active ingredient particles, including any solid support, binder or other pharmaceutically acceptable excipient, with at least one taste-masking layer. The thickness of the taste-masking layer(s) is/are generally from about 2% to about 100%. Preferably, the thickness is from about 5% to about 90%, more preferably from about 10% to about 80%, and most preferably, from about 15% to about 50%. Alternatively, the thickness of the taste-masking layer(s) can be expressed in terms of the percentage of the coating in the taste-masked particles. These two alternative expressions of coating thickness can be easily translated from one to another. It will be appreciated that the upper limit of the coating thickness is affected by the effect it can have on the dissolution of the drug and by practical considerations like cost, weight, size, volume, etc. The lower limit of coating thickness is heavily dictated by its taste-masking ability. Preferably, the coating should substantially cover the entire surface area of the active ingredient particles. In addition to the water-soluble and water-insoluble polymers, the taste-masking layer can include other ingredients.

In yet another preferred embodiment of the present invention, the coated active ingredient particles contain up to 80% of the at least one active ingredient, preferably from about 0.5% to about 50%, and more preferably from about 2% to about 35%.

A advantage of the taste-masked formulation of the present invention is that it can effectively mask the taste of highly water-soluble drugs, and in particular, cetirizine hydrochloride, and at the same time, provide rapid dissolution of the active ingredient in an acidic medium, typically the patient's stomach.

By the term “effectively masks the taste,” and like terms it is meant that the active ingredient is coated in such a way that is sufficient to prevent the dissolution of at least the majority of the active ingredient in a patient's mouth for some defined period of time. The nature of the coating will vary depending upon such factors as the solubility of the particular active ingredient, the taste of the active ingredient and the desired level of taste-masking effect. In a preferred embodiment, “effectively masks the taste” requires that that the active ingredient is coated such that less than about 20% of the active ingredient in the formulation is released within three minutes under basic pH (e.g., in the mouth), and at least about 65% of the active ingredient is released in acidic pH within thirty minutes.

As used herein, basic pH includes a pH of as low as about 6.0 and acidic pH includes a pH of less than 6.0.

The term “highly water-soluble” means that the active ingredient in the formulation has a solubility of at least about 0.05 g/ml in an aqueous solution at room temperature, preferably at least about 1.0 g/ml, more preferably at least about 1.5 g/ml and the most preferably at least about 1.6 g/ml. The term “room temperature” typically ranges from about 10° C. to about 30° C., preferably from about 15° C. to about 25° C.

The present invention can be used for any active pharmaceutical ingredients, whether in need of taste-masking or not. However, where the underlying active ingredient does not need taste-masking, there may be no need to resort to the added steps of the present invention. For particularly poor tasting drugs, such as cetirizine hydrochloride, where any significant exposure of the active ingredient within the patient's mouth may be unacceptable, the present invention is particularly valuable.

Without intending to be bound by any theory of operation, it is believed that the dissolution profile is affected by at least two variables, the weight ratio of water-soluble polymer and water-insoluble polymer in the coating and the thickness of the coating. Other variables which can affect the dissolution profile include the solubility of the active ingredient, the diameters of the support, if any, the tablet size, the tablet hardness, specific surface area of the coated active ingredient particles, materials used, etc.

As used herein, “active-ingredients” include those having a water solubility of about 0.03 g/ml or more. Active ingredients in accordance with the present invention can be any pharmaceutically active ingredient including, without limitation, abortifacient/interceptive, ace-inhibitor, α-adrenergic agonist, β-adrenergic agonist, α-adrenergic blocker, β-adrenergic blocker, adrenocortical steroid, adrenocortical suppressant, adrenocorticotropic hormone, alcohol deterrent, aldose reductase inhibitor, aldosterone antagonist, 5-alpha reductase inhibitor, anabolic, analeptic, analgesic, androgen, angiotensin converting enzyme inhibitor, angiotensin II receptor antagonist, anorexic, antacid, anthelmintic, antiacne, antiallergic, antialopecia agent, antiamebic, antiandrogen, antianginal, antiarrhythmic, antiarteriosclerotic, antiarthritic/antirheumatic, antiasthmatic, antibacterial, antibacterial adjuncts, antibiotic, anticancer, anticholelithogenic, anticholesteremic, anticholinergic, anticoagulant, anticonvulsant, antidepressant, antidiabetic, antidiarrheal, antidiuretic, antidote, antidyskinetic, antieczematic, antiemetic, antiepileptic, antiestrogen, antifibrotic, antiflatulent, antifungal, antiglaucoma, antigonadotropin, antigout, antihemorrhagic, antihistaminic, antihypercholesterolemic, antihyperlipidemic, antihyperlipoproteinemic, antihyperphosphatemic, antihypertensive, antihyperthyroid, antihypotensive, antihypothyroid, anti-infective, anti-inflammatory, antileprotic, antileukemic, antilipemic, antimalarial, antimanic, antimethemoglobinemic, antimigraine, antimycotic, antinauseant, antineoplastic, antineoplastic adjunct, antineutropenic, antiosteoporotic, antipagetic, antiparkinsonian, antiperistaltic, antipheochromocytoma, antipneumocystis, antiprostatic hypertrophy, antiprotozoal, antipruritic, antipsoriatic, antipsychotic, antipyretic, antirheumatic, antirickettsial, anti seborrheic, anti septic/disinfectant, antispasmodic, antisyphilitic, antithrombocythemic, antithrombotic, antitubercular, antitumor, antitussive, antiulcerative, antiurolithic, antivenin, antivertigo, antiviral, anxiolytic, aromatase inhibitors, astringent, benzodiazepine antagonist, beta-blocker, bone resorption inhibitor, bradycardic agent, bradykinin antagonist, bronchodilator, calcium channel blocker, calcium regulator, calcium supplement, cancer chemotherapy, capillary protectant, carbonic anhydrase inhibitor, cardiac depressant, cardiotonic, cathartic, CCK antagonist, central stimulant, cerebral vasodilator, chelating agent, cholecystokinin antagonist, cholelitholytic agent, choleretic, cholinergic, cholinesterase inhibitor, cholinesterase reactivator, CNS stimulant, cognition activator, contraceptive, control of intraocular pressure, converting enzyme inhibitor, coronary vasodilator, cytoprotectant, debriding agent, decongestant, depigmentor, dermatitis herpetiformis suppressant, diagnostic aid, digestive aid, diuretic, dopamine receptor agonist, dopamine receptor antagonist, ectoparasiticide, emetic, enkephalinase inhibitor, enzyme, enzyme cofactor, enzyme inducer, estrogen, estrogen antagonist, expectorant, fibrinogen receptor antagonist, gastric and pancreatic secretion stimulant, gastric proton pump inhibitor, gastric secretion inhibitor, gastroprokinetic, glucocorticoid, α-glucosidase inhibitor, gonad-stimulating principle, gout suppressant, growth hormone inhibitor, growth hormone releasing factor, growth stimulant, hematinic, hematopoietic, hemolytic, hemostatic, heparin antagonist, hepatoprotectant, histamine H₁-receptor antagonist, histamine H₂-receptor antagonist, HIV proteinase inhibitor, HMG CoA reductase inhibitor, hypnotic, hypocholesteremic, hypolipidemic, hopotensive, immunomodulator, immunosuppressant, intropic agent, insulin sensitizer, ion exchange resin, keratolytic, lactation stimulating hormone, laxative/cathartic, leukotriene antagonist, LH-RH agonist, lipotropic, 5-lipoxygenase inhibitor, lupus erythematosus suppressant, major tranquilizer, matrix metalloproteinase inhibitor, mineralocorticoid, minor tranquilizer, miotic, monoamine oxidase inhibitor, mucolytic, muscle relaxant, mydriatic, narcotic analgesic, narcotic antagonist, nasal decongestant, neuroleptic, neuromuscular blocking agent, neuroprotective, nootropic, nsaid, opioid analgesic, oral contraceptive, ovarian hormone, oxytocic, parasympathomimetic, pediculicide, pepsin inhibitor, peripheral vasodilator, peristaltic stimulant, pigmentation agent, plasma volume expander, potassium channel activator/opener, pressor agent, progestogen, prolactin inhibitor, prostaglandin/prostaglandin analog, protease inhibitor, proton pump inhibitor, pulmonary surfactant, 5α-reductase inhibitor, replenishers/supplements, respiratory stimulant, retroviral protease inhibitor, reverse transcriptase inhibitor, scabicide, sclerosing agent, sedative/hypnotic, serenic, serotonin noradrenaline reuptake inhibitor, serotonin receptor agonist, seratonin receptor antagonist, serotonin uptake inhibitor, skeletal muscle relaxant, somatostatin analog, spasmolytic, stool softener, succinylcholine synergist, sympathomimetic, thrombolytic, thromboxane A₂-receptor antagonist, thromboxane A₂-sythetase inhibitor, thyroid hormone, thyroid inhibitor, thyrotropic hormone, tocolytic, topical protectant, topoisomerase I inhibitor, topoisomerase II inhibitor, tranquilizer, ultraviolet screen, uricosuric, vasodilator, vasopressor, vasoprotectant, vitamin/vitamin source, vulnerary, Wilson's disease treatment, xanthine oxidase inhibitor. Preferably, the drug is selected from the group consisting of acyclovir; auranofin; bretylium; cytarabine; doxepin; doxorubicin; hydralazine; ketamine; labetalol; mercaptopurine; methyldopa; nalbuphine; nalozone; pentoxifyll; pyridostigmine; terbutaline; verapamil; buserelin; calcitonin; cyclosporin; oxytocin and heparin. More preferably, the active ingredient is the antihistamine cetirizine hydrochloride, diphenhydramine hydrochloride, chlorpheniramine maleate, pseudoephedrine hydrochloride.

In general, the predetermined amount of active ingredient incorporated into each formulation may be selected according to known principles of pharmacy. “Formulation” means an amount of active ingredient and pharmaceutically acceptable excipients combined together which are ultimately incorporated into an overall dosage form. Generally, the amount of active ingredient incorporated is a pharmaceutically effective amount. A “pharmaceutically effective amount” is the amount or quantity of an active ingredient which is sufficient to elicit the required or desired therapeutic response. In other words, it is the amount which is sufficient to elicit an appreciable biological response when administered to a patient. Of course, the amount of active ingredient used can vary greatly. It depends on the size of the dosage, the requirements of other ingredients, the size, age, weight, sex, condition of the patient, their medical condition, and the number of, for example, tablets which constitute a single dose. Typically, an active ingredient in each dosage form can be present in an amount of from about 0.1 mg to about 1000 mg, preferably from about 1 mg to about 500 mg and more preferably from about 4 mg to about 200 mg. Conventional amount of pharmaceutically acceptable excipients can be used in this these formulations as well. Typically, pharmaceutically acceptable excipients can be used in an amount about 5% to about 90% by weight, based on the weight of the dosage form (pharmaceutically acceptable excipients and coated active ingredient particles). More preferably 10%-50% of said pharmaceutically acceptable excipients can be present.

The active ingredient may be used in any particulate form, such as powders, crystals, amorphous particles, granules, spheroid particles, agglomerates, liquid capsule, liquid adsorbed on a solid particles and the like. They may also include solid supports such as a powder, adsorbent, granule, bead, sphere, and the like. Once the desired amount of active ingredient is selected, an amount of solid support sufficient to allow the amount of the active ingredient to be applied thereon is selected and provided.

More than one active ingredient may be contained in one or more layers. The active ingredient is applied or preferably uniformly layered or deposited onto the beads through a process such as fluid bed coating, coating in a coating pan, spray coating, spray congealing, or coacervation. Preferably, the active ingredient layer is dried before applying the taste-masking layer.

The taste-masking layer is then coated, absorbed or adsorbed to the active ingredient layer and should substantially completely surround them. The taste-masking layer can be prepared from mixing a water-soluble polymer and a water-insoluble polymer. The amounts of water-soluble polymer and water-insoluble polymer are selected so that effective taste-masking is achieved as well as achieving the desired dissolution profile.

Various dosage forms can be prepared from the taste-masked formulation and at least one pharmaceutically acceptable excipient, which includes, but is not limited to binders, fillers, lubricants, effervescent and/or non-effervescent disintegrants, super disintegrants bulking agents, colors, solvents, flavors adsorbents or absorbants, and the like. Pharmaceutically acceptable excipients also include those disclosed in Authur H. Kibbe, HandBook of Pharmaceutical Excipients, 3d Ed, the content of which is hereby incorporated by reference to the extent permitted. “Dosage form” includes, but is not limited to orally disintegrating tablets or capsule, soft gel capsule, caplet, slugged capsule, chewable tablet and the like. These dosage forms can be prepared using techniques known in the art. Moreover, additional ingredients, such as disintegrants, binders, lubricants, can be added to the dosage form. Typically, pharmaceutically acceptable excipients can be used in an amount about 5% to about 90% by weight, based on the weight of the dosage form (pharmaceutically acceptable excipients and coated active ingredient particles). More preferably 10%-50% of said pharmaceutically acceptable excipients can be present.

To exemplify the concepts described above, various experiments were carried out to measure the dissolution profiles of various formulations. However, these examples are intended to further illustrate certain preferred embodiments of the invention and are not limiting in nature and are not intended to limit the invention in any way or to set forth specific active ingredients, water-soluble polymers, water-insoluble polymers, binders, preparation and testing procedures, or other parameters which must be used exclusively to practice the invention. Hence, for example the use of cetirizine to illustrate aspects of water-soluble drugs as a whole is purely for illustrative purposes and should not be construed as limiting the invention.

Further, any range of numbers recited in the specification or paragraphs hereinafter describing or claiming various aspects of the invention, such as that representing a particular set of properties, units of measure, conditions, physical states or percentages, is intended to literally incorporate expressly herein by reference or otherwise, any number falling within such range, including any subset of numbers or ranges subsumed within any range so recited. The term “about” when used as a modifier for, or in conjunction with, a variable, is intended to convey that the numbers and ranges disclosed herein are flexible and that practice of the present invention by those skilled in the art using temperatures, concentrations, amounts, contents, and properties that are outside of the range or different from a single value, will achieve the desired result, namely, a taste-masked formulation and methods for preparing and using such formulations.

### EXAMPLE 1

### Preparation of Cetirizine Formulation

Taste-masked particles containing cetirizine which satisfy the criteria of the present invention were prepared by (1) mixing 20.4 grams of cetirizine HCl, 2.9 grams of polyvinyl pyrrolidone and 5.8 grams of hydroxypropyl methylcellulose, (2) coating the mixture of step (1) onto 50 grams of microcrystalline cellulose spheres, having a diameter of 230 micrometer using a fluid bed coater, and then dried. (3) mixing 18 grams of Surelease® (ethylcellulose produced by Colorcon, Lot Number E719010) and 2.0 grams of Opadry® (HPMC produced by colorcon, Lot Number YF119053) and (4) coating the mixture of step (3) onto the cetirizine coated microcrystalline cellulose spheres of step (2).

TABLE 1

| COMPONENT NAME | QUANTITY (g) |
|---|---|
| Cetirizine HCl | 20.4 |
| Hydroxypropyl Methylcellulose | 5.8 |
| Polyvinyl Pyrrolidone | 2.9 |
| Microcrystalline Cellulose Spheres | 50.9 |
| Surelease ® (ethylcellulose), Clear | 18.0 |
| Opadry ® (HPMC), Clear | 2.0 |
| Total | 100.0 |

Another batch of taste-masked particles in accordance with the present invention were made as follows:

500 g of cetirizine layered beads were prepared by coating cetirizine to the beads. A coating solution of 833.3 g was also prepared by mixing 450 g of Surelease Clear containing 25% solids and 12.5 g of Opadry with 370.8 g of water. Then, the coating solution is coated onto the 500 g of cetirizine layered beads and the coated beads were dried. The 833.3 g of coating solution on the 500 g of cetirizine layered beads represents a coating thickness of about 20%.

### EXAMPLE 2

### Testing the Effectiveness of the Coatings Containing 80% Water-Insoluble and 20% Water-Soluble Polymer Mixture on Masking the Taste of Cetirizine

Cetirizine formulations were prepared generally in the same way as that described in Example 1. In addition, the coated beads were compressed into tablets. The tablet size is 300 mg and ⅜ inch. The tablets contain 23.6% of the coated drug, about 56% of mannitol, 5% of aspartame, 5% of crospovidone, 3% of microcrystalline cellulose, 2% of citric acid, 3% of sodium bicarbonate, 0.4% of flavor, 0.4% of color, 0.3% of silicon dioxide, and 1.5% of magnesium stearate. The tablets have hardness of about 30 Newtons. Tablets compressed from the coated spheres were subjected to dissolution testing. The dissolution studies for both pH 7.3 and pH 2.0 were done according to the USP paddle method. see UPS Dissolution Apparatus 2 (U.S. Pat. No. 24:1942). The volume of the dissolution medium was 900 ml. The dissolution test results of these formulations are listed in Table 2.

TABLE 2
(80% Water-Insoluble Polymer/20% Water-Soluble Polymer)

| Coating Thickness | | Coating Thickness | |
|---|---|---|---|
| 35% | | 40% | |
| pH 7.3 | 0.01N HCl | pH 7.3 | 0.01N HCl |
| 3 min | 30 min | 3 min | 30 min |
| 9% | 73% | 9% | 69% |

¹Simulated mouth conditions; phosphate buffer was used to control the solution pH at 7.3.
²Simulated stomach conditions.
³Measured time since administration in minutes (+/−10 seconds).
#coating thickness of 35% and 40% refer to weight gains of 35% and 40%, respectively.

Table 2 shows the results of dissolution experiments for tablets prepared from the coated spheres containing a ratio of 80% Surelease® (ethylcellulose) and 20% Opadry® (HPMC) at various coating thicknesses. For each coating thickness, the time and percent dissolution is reported for both basic (pH 7.3) and acidic (0.01N HCl, pH 2.0) conditions. Each of these coating thicknesses meets the criteria that the release of the active ingredient is less than 20% in aqueous solution at a neutral or basic pH in 3 minutes and the release of the active ingredient is more than about 65% in acidic pH in 30 minutes.

### EXAMPLE 3

### Testing the Effectiveness of the Coatings Containing 85% Water-Insoluble and 15% Water-Soluble Polymer Mixture on Masking the Taste of Cetirizine

Cetirizine formulations were also prepared in the same way as that described in Example 2, except the ratio of water-insoluble and water-soluble polymers. The dissolution test results of these formulations are listed in Table 3.

TABLE 3
(85% Water-Insoluble Polymer/15% Water-Soluble Polymer)

| Coating Thickness | | Coating Thickness | | Coating Thickness | |
|---|---|---|---|---|---|
| 20% | | 25% | | 30% | |
| pH 7.3¹ | 0.01N HCl | pH 7.3 | 0.01N HCl | pH 7.3 | 0.01N HCl |
| 3 min | 30 min 95% | 3 min | 30 min 79% | 3 min | 30 min 69% |
| 16% | | 9% | | 3% | |

¹Simulated mouth conditions; phosphate buffer was used to control the solution pH at 7.3.

Table 3 shows the measured results of dissolution tests for tablets prepared from the coated spheres containing a ratio of about 85% Surelease® (ethylcellulose) and about 15% Opadry (HPMC) at various coating thicknesses. For each coating thickness, the time and percent dissolution is reported for both basic (pH 7.3) and acidic (0.01 N HCl) conditions. A review of the data in Table 3 reveals that all three coating thicknesses meet the performance criteria.

### EXAMPLE 4

### Testing the Effectiveness of the Coatings Containing 90% Water-Insoluble and 10% Water-Soluble Polymer Mixture on Masking the Taste of Cetirizine

Cetirizine formulations were prepared in the same way as that described in Example 2, except the ratio of water-soluble and water-insoluble polymers. The dissolution test results of these formulations are listed in Table 4.

TABLE 4
(90% Water-Insoluble Polymer/10% Water-Soluble Polymer)

| Coating Thickness 20% | | Coating Thickness 25% | |
|---|---|---|---|
| pH 7.3 | 0.01 N HCl | pH 7.3 | 0.01 N HCl |
| 3 min 16% | 130 min 83% | 3 min 7% | 30 min 74% |

Table 4 shows the results of dissolution tests for coated tablets containing about 90% Surelease® (ethylcellulose) and about 10% Opadry® (HPMC) at coating thicknesses of 20% and 25%. For each coating thickness, the time and percent dissolution are given for both basic (pH 7.3) and acidic (0.01 N HCl) conditions. The data in Table 4 reveal that of the two thicknesses tested, both yield a desirable dissolution profile while simultaneously providing a more desirable taste-masking effect.

The principles, preferred embodiments, and modes of operation of the present invention have been described in the foregoing specification. The invention which is intended to be protected herein, however, is not to be construed as limited to the particular forms disclosed, since these are to be regarded as illustrative rather than restrictive. Variations and changes may be made by those skilled in the art, without departing from the spirit of the invention.

1. A taste-masked formulation comprising: taste-masked particles including (1) at least one active ingredient and (2) a taste-masking layer surrounding said at least one active ingredient, said taste-masking layer including from about 2% to about 20% of a water-soluble polymer and from about 80% to about 98% of a water-insoluble polymer, said water-insoluble polymer being selected from the group consisting of ethyl cellulose, cellulose acetate phthalate, hydroxypropyl methyl cellulose phthalate, polyvinyl acetate phthalate, hydroxypropyl methyl cellulose succinate, and shellac, and wherein said taste-masking layer has a coating thickness from about 2% to about 100% by weight, based on the weight of said taste-masked particles, wherein said taste-masked formulation releases less than about 20% of the at least one active ingredient in an aqueous solution at a basic pH in about 3 minutes and releases at least about 65% of said at least one active ingredient at an acidic pH in about 30 minutes.

2. The taste-masked formulation of claim 1, wherein said release is less than about 16% of the at least one active ingredient in an aqueous solution at a neutral to basic pH in about 3 minutes and releases at least about 75% of said at least one active ingredient at an acidic pH in about 30 minutes.

3. The taste-masked formulation of claim 1, wherein said water-soluble polymer is selected from the group consisting of hydroxypropyl methyl cellulose, polyvinyl pyrrolidone, polyvinyl alcohol, hydroxypropyl cellulose, hydroxyethyl cellulose, methyl cellulose, carboxymethyl cellulose, and polyethylene glycols.

4. The taste-masked formulation of claim 1, wherein said taste-masking layer includes from about 5% to about 15% of said water-soluble polymer and from about 85% to about 95% of said water-insoluble polymer.

5. The taste-masked formulation of claim 1, wherein said taste-masking layer includes from about 10% to about 15% of said water-soluble polymer and from about 85% to about 90% of said water-insoluble polymer.

6. The taste-masked formulation of claim 1, wherein said taste-masking layer is provided in an amount from about 25% to about 100% of the taste-masked particles by weight.

7. The taste-masked formulation of claim 1, wherein said taste-masking layer is provided in an amount from about 30% to about 100% of the taste-masked particles by weight.

8. The taste-masked formulation of claim 1, further comprising a solid support in intimate contact with said active ingredient.

9. The taste-masked formulation of claim 1, wherein said solid support is in the form of particles.

10. The taste-masked formulation of claim 9, wherein said particles are in the form of beads, or spheres.

11. The taste-masked formulation of claim 9, wherein the particles are in the form of spheres which are selected from the group consisting of sugar spheres, microcrystalline cellulose spheres.

12. The taste-masked formulation of claim 1, further comprising a binder which facilitates the binding of said active ingredient onto said solid support.

13. The taste-masked formulation of claim 1, wherein said at least one active ingredient has a water solubility of about 0.03 g/ml or more.

14. The taste-masked formulation of claim 1, wherein said at least one active ingredient has a water solubility of at least 1.0 g/ml at room temperature.

15. The taste-masked formulation of claim 1, wherein said at least one active ingredient has a water solubility of at least 1.5 g/ml at room temperature.

16. The taste-masked formulation of claim 1, wherein said at least one active ingredient has a water solubility of at least 1.6 g/ml at room temperature.

17. The taste-masked formulation of claim 1, wherein said at least one active ingredient is selected from the group consisting of cetirizine hydrochloride and pseudoephedrine.

18. The taste-masked formulation of claim 12, wherein said binder is selected from the group consisting of: hydroxypropyl methylcellulose, HPMC, polyvinyl pyrrolidone, starch, gum arabic, carboxymethylcellulose, polyvinylpyrrolidone, hydroxypropylcellulose, crystalline cellulose, sucrose, D-mannitol, dextrin, hydroxypropyl cellulose, hydroxypropylmethyl cellulose, polyvinylpyrrolidone, povidone, acacia, tragacanth, gelatin, cellulose materials, alginic acids and salts thereof, magnesium aluminum silicate, polyethylene glycol, guar gum, polysaccharide acids, bentonites, sugars, and invert sugars.

19. The taste-masked formulation of claim 16, wherein the cellulose materials are methyl cellulose or sodium carboxy methyl cellulose.

20. The taste-masked formulation of claim 1, wherein said formulation further comprises at least one pharmaceutically acceptable excipient.

21. A pharmaceutical dosage form comprising a taste-masked formulation which includes: taste-masked particles including (1) at least one active ingredient and (2) a taste-masking layer surrounding said at least one active ingredient, said taste-masking layer including from about 2% to about 20% of a water-soluble polymer and from about 80% to about 98% of a water-insoluble polymer, said water-insoluble polymer being selected from the group consisting of ethyl cellulose, cellulose acetate phthalate, hydroxypropyl methyl cellulose phthalate, polyvinyl acetate phthalate, hydroxypropyl methyl cellulose succinate, and shellac, and wherein said taste-masking layer is provided in an amount of at least 10% of said taste-masked particles by weight, wherein said taste-masked formulation releases less than about 20% of the at least one active ingredient in an aqueous solution at a basic pH in about 3 minutes and releases at least about 65% of said at least one active ingredient at an acidic pH in about 30 minutes.

22. The pharmaceutical dosage form of claim 21, wherein said dosage form is selected from the group consisting of orally disintegrating tablet, orally disintegrating capsule, soft gel capsule, caplet, slugged capsule, and chewable tablet.

23. The pharmaceutical dosage form of claim 21, further comprising at least one pharmaceutically acceptable excipient.

24. The pharmaceutical dosage form of claim 22, further comprising a binder, a filler, a lubricant, a disintegrant, a bulking agent, a color, a solvent, a flavor adsorbent or a flavor absorbent.

25. The pharmaceutical dosage form of claim 21, wherein said pharmaceutically acceptable excipient is used in an amount from about 5% to about 90% by weight based on the weight of the dosage form.

26. The pharmaceutical dosage form of claim 21, wherein the at least one active ingredient is present in an amount from about 0.1 mg to about 1000 mg per dosage.

27. The pharmaceutical dosage form of claim 26, wherein the at least one active ingredient is present in an amount from about 1 mg to about 500 mg per dosage.

28. The pharmaceutical dosage form of claim 26, wherein the at least one active ingredient is present in an amount from about 4 mg to about 200 mg per dosage.

29. The pharmaceutical dosage form of claim 26, wherein said taste-masking layer is provided in an amount from about 10% to about 100% of said taste-masked particles by weight.

30. The pharmaceutical dosage form of claim 25, wherein the at least one active ingredient is present in an amount from about 0.1 mg to about 1000 mg per dosage.
